(19) United States
(12) Patent Application Publication
(10) Pub. No.: US 2024/0023879 A1
(43) Pub. Date: Jan. 25, 2024

(54) A COMPUTER-IMPLEMENTED MODEL FOR PREDICTING OCCURRENCE OF A SEIZURE AND TRAINING METHOD THEREOF

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE - CNRS, PARIS (FR); SORBONNE UNIVERSITE, 75006 (FR); INSTITUT DU CERVEAU ET DE LA MOELLE EPINIERE – ICM, Paris (US); ASSISTANCE PUBLIQUE HOPITAUX DE PARIS, Paris (FR)

(72) Inventors: Mario CHAVEZ, Paris (FR); Louis COUSYN, PARIS (FR); Vincent NAVARRO, PARIS (FR)

(21) Appl. No.: 18/256,800

(22) PCT Filed: Dec. 10, 2021
(86) PCT No.: PCT/EP2021/085146

§ 371 Date: Jun. 9, 2023

(30) Foreign Application Priority Data
Dec. 11, 2020 (EP) 20306548.7

(51) Int. Cl. A61B 5/00; G16H 50/20

(52) U.S. Cl. CPC A61B 5/4094; A61B 5/7267; A61B 5/746; G16H 50/20

(57) ABSTRACT

The invention relates to a method for training a model for predicting occurrence of an epileptic seizure, the method comprising performing a supervised training over a training dataset of a nonlinear binary classification model configured to receive as input the evaluation, by a patient, of the intensity of each prodromal symptom among a predefined set of prodromal symptoms, and to output a classification of said patient belonging either to a pre-ictal or inter-ictal state, and the training dataset comprises data inputs obtained from a plurality of epileptic patients, each data input comprising an evaluation, by a patient, of the intensity of each of the predefined set of prodromal symptoms, each data input being further associated to an indication of said patient belonging to a pre-ictal or inter-ictal state at the time of the evaluation. The invention also relates to a prediction model obtained accordingly, and a computing device for implementing said prediction model.

### FIELD OF THE INVENTION

The present application relates to the prediction of epileptic seizures based on prodromal symptoms experienced by epileptic patients, using a machine learning approach.

### BACKGROUND OF THE INVENTION

Epileptic seizures have long been considered as resulting from an abrupt and unpredictable transition in brain activity. However, recent studies on the underlying mechanisms of transition from an interictal state—being a state between two seizures—and a seizure, suggest preictal changes, i.e. changes happening before the actual seizure. It is for instance the case of the publication by Kuhlmann L. et al, “Seizure prediction, ready for a new era”, Nat Rev Neurol. 2018 Oct. 14 (10) 618-30. A reliable identification of a high-risk state of seizure before the seizure actually happens may yield a time-window for a broad range of therapeutic possibilities. Treatment modalities could then vary from preventive measures, such as avoiding potentially risky activities (driving, handling heavy machines), or designing behavioral and/or preventive treatments.

Seizures can be preceded by subjective symptoms that are interpreted by patients as precursors of an upcoming seizure. These prodromal symptoms can be of various types, and their intensity and nature may vary from one patient to another. Prodromal symptoms are poorly understood but may reflect changes in the brain activity related to the preictal period. Some prospective studies investigated the ability of prodromal symptoms in seizure prediction, and highlighted premonitory features but with rather low sensitivities.

In particular, a recent publication by Privitera M. et al, “Seizure self-prediction in a randomized controlled trial of stress management” Neurology 2019; 93(22): e202 provides the results of a study evaluating the statistical significance of a number of prodromal symptoms including:
Seizure self-prediction using a five-point Likert scale, associated with seizure occurrence at 6, 12 and 24 hours following the patients filling the questionnaire,
0-100 visual analog scales of 4 mood valences,
Absence or presence of 11 premonitory symptoms, and
Number of hours of sleep.

The results of this study show, for the 12-hour prediction window, median specificity for seizure prediction of 0.94 and median sensitivity of 0.10. This low sensitivity value does not enable to implement a prediction method based on this study. A number of other studies have been performed on this topic, the results of which are summarized up in Table 1 below. It appears that none of these studies provided a fully reliable method to predict a seizure based on prodromal symptoms.

TABLE 1

| | Study | Nb | | Statistical | |
|---|---|---|---|---|---|
| Ref | design | patients | Prodromal symptoms | method | Results |
| (1) | Daily | 83 | Seizure self- | Generalized | Twofold increase in |
| | questionnaire, | | prediction in the | mixed-effects | seizures following a |
| | VEEG | | next 24 hours (yes/ | model | positive prediction |
| | monitoring | | no/do not know) | | |
| (2) | Diary, | 71 | Seizure self- | Odds | Positive prediction was |
| | outpatient | | prediction in the | Ratio | associated with a twofold |
| | | | next 24 hours using | and Chi | increased risk of seizure |
| | | | a four-point Likert | square | (Odds Ratio 2.25); |
| | | | scale | test | Specificity 0.87; |
| | | | | | Selectivity 0.21 |
| (3) | Diary, | 71 | Same as (2) Hours | Odds | One-unit increments of |
| | outpatient | | of sleep | Ratio | stress and anxiety were |
| | | | 0-10 scales for | and logit | associated with an |
| | | | anxiety and stress | normal | increased risk of seizure |
| | | | | multiple | the following day |
| | | | | logistic | Increased hours of sleep |
| | | | | regression | were associated with a |
| | | | | model | reduced risk of seizures |
| | | | | | Self-prediction (Odds |
| | | | | | Ratio 3.7) and hours of |
| | | | | | sleep for the night prior |
| | | | | | to the seizure remained |
| | | | | | significant in multiple |
| | | | | | logistic regression model |
| (4) | e-diary, | 19 | 0-100 visual analog | Odds | Several mood items and |
| | outpatient | | scales: happy, sad, | Ratio | 10 premonitory features |
| | | | relaxed, nervous, | and | associated with increased |
| | | | lively, bored | multivariate | odds of seizure |
| | | | 0-10 scale: stress | logistic | In multivariate models, a |
| | | | Premonitory | regression | 10-point improvement in total |
| | | | features: 18 items | model | mood decreased seizure risk |
| | | | (yes/no) | | by 25% while each additional |
| | | | Hours of sleep | | significant premonitory |
| | | | | | feature increased seizure |
| | | | | | risk by nearly 25% |
| (5) | e-diary, | 19 | Seizure self- | Odds | Odds Ratio for prediction |
| | outpatient | | prediction in the next | Ratio | choices within 6 h was as high |
| | | | 24 hours using a | and | as 9.31 for “almost certain” |
| | | | five-point Likert scale | multivariate | For 9 best predictors, median |
| | | | 0-100 visual analog | logistic | sensitivity of self-prediction was |
| | | | scales: happy, sad, | regression | 0.5 and median specificity 0.95 |
| | | | relaxed, nervous, | model | In multivariate models, self- |
| | | | lively, bored | | prediction, favorable change in |
| | | | 0-10 scale: stress | | mood and number of |
| | | | Premonitory | | premonitory symptoms were |
| | | | features: 18 items | | significant |
| | | | (yes/no) | | |
| | | | Hours of sleep | | |
| (6) | PDA, | 9 | Entries of present | Selectivity, | No significant result |
| | outpatient | | prodromal symptoms | Specificity | |
| | | | (not specified in the | | |
| | | | paper) | | |

The references referred to in this Table are as follows:
1. DuBois J M, Boylan L S, Shiyko M, Barr W B, Devinsky O. Seizure prediction and recall. Epilepsy Behav. 2010 May; 18(1-2):106-9.
2. Haut S R, Hall C B, LeValley A J, Lipton R B. Can patients with epilepsy predict their seizures? Neurology. 2007 Jan. 23; 68(4):262-6.
3. Haut S R, Hall C B, Masur J, Lipton R B. Seizure occurrence: Precipitants and prediction. Neurology. 2007 Nov. 13; 69(20):1905-10.
4. Haut S R, Hall C B, Borkowski T, Tennen H, Lipton R B. Clinical features of the pre-ictal state: Mood changes and premonitory symptoms. Epilepsy Behav. 2012 April; 23(4):415-21.
5. Haut S R, Hall C B, Borkowski T, Tennen H, Lipton R B. Modeling seizure self-prediction: An e-diary study. Epilepsia. 2013 November; 54(11):1960-7.
6. Maiwald T, Blumberg J, Timmer J, Schulze-Bonhage A. Are prodromes preictal events? A prospective PDA-based study. Epilepsy Behav. 2011 June; 21(2):184-8.

There is therefore a need for a prediction method enabling more accurate prediction of epileptic seizure from prodromal symptoms.

### PRESENTATION OF THE INVENTION

In view of the above, the invention aims at proposing a model for reliably predicting occurrence of epileptic seizures based on prodromal symptoms.

Another aim of the invention is to allow a patient to obtain regularly an indication of a risk of epileptic seizures in a determined period of time to follow.

Another aim of the invention is to propose a model which can be personalized for a given patient.

In view of the above, a method for training a model for predicting occurrence of an epileptic seizure is disclosed, the method being implemented by a training device comprising a computer and a memory storing a training dataset,
wherein the training dataset comprises data inputs obtained from a plurality of epileptic patients, each data input comprising an evaluation, by a patient, of the intensity of each of a predefined set of prodromal symptoms, each data input being further associated to an indication of said patient belonging to a pre-ictal or inter-ictal state at the time of the evaluation,
wherein a pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation,
and the method comprises performing supervised training, over the training dataset, of a nonlinear binary classification model configured to receive as input the evaluation, by a patient, of the intensity of each prodromal symptom among the predefined set of prodromal symptoms, and to output a classification of said patient belonging either to a pre-ictal or inter-ictal state.

In embodiments, the intensity of each prodromal symptom is evaluated on a scale comprising between two and ten points, preferably between three and ten points. For instance, the intensity of each prodromal symptom may be evaluated on a four-points Likert scale.

In embodiments, the fixed period of time following the evaluation may be between 6 h and 24 h.

In embodiments, the prediction model is a SVM classifier with a Gaussian kernel.

In embodiments, the method may further comprise a step of selecting, among the predefined set of prodromal symptoms, a subset of prodromal symptoms maximizing the performances of the model over the training dataset.

In embodiments, the method may further comprise a step of selecting, among the predefined set of prodromal symptoms, a subset of prodromal symptoms maximizing the performances of the model over the data inputs relative to a specific patient.

In embodiments, the model is a SVM classifier and training of the SVM classifier comprises determining a hyperplane separating input data into two classes corresponding respectively to a pre-ictal state and an inter-ictal state, and the SVM classifier is further configured to output an indication of a distance between each piece of input data and said hyperplane.

According to another object, a training device of a model for predicting epileptic seizure is disclosed, comprising a computer and a memory storing a training dataset,
wherein the training dataset comprises data inputs obtained from a plurality of epileptic patients, each data input comprising an evaluation, by a patient, of the intensity of each of a predefined set of prodromal symptoms, and an indication of said patient belonging to a pre-ictal or inter-ictal state at the time of the evaluation,
wherein a pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation,
the training device being characterized in that it is configured for implementing the method according to the above description.

According to another object, it is also disclosed a computer-implemented Support Vector Machine model, configured to receive as input an evaluation by a patient of the intensity of each one of a set of prodromal symptoms, and to output an indication of the patient belonging to a pre-ictal state or inter-ictal state, wherein the pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation,
characterized in that it is trained by implementing the method according to the description above.

According to another object, it is also disclosed a computer-implemented comprising:
implementing a model trained according to the above method on an evaluation by a patient of the intensity of each one of a set of prodromal symptoms, to output an indication of the patient belonging to a pre-ictal or inter-ictal state, wherein a pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation.

In embodiments, this method may further comprise a preliminary step of receiving the evaluation by the patient of the intensity of each one of the set of prodromal symptoms.

In embodiments, the steps of receiving an evaluation and implementing the model are performed regularly with a time interval between two evaluations corresponding to said fixed period of time.

In embodiments, the model is a SVM classifier which is configured to output an indication of a distance between each piece of input data and a hyperplane separating input data into two classes corresponding respectively to a pre-ictal state and an inter-ictal state, and the method further comprises computing a confidence index associated to the classification of the patient as belonging to the pre-ictal or inter-ictal state, based on the distance between a vector representative of the patient's evaluation and the hyperplane determined during training of the model, and outputting said confidence index.

Another object of the present disclosure is a computer-program product comprising code instructions for executing one of the methods disclosed above, when it is implemented by a computer.

Another object of the present disclosure is a computing device comprising a processing unit, a memory storing code instructions executed by the processing unit, and a Human-Machine Interface, wherein the computing device is configured for:
receiving, from a patient, an evaluation of the intensity of each of a plurality of prodromal symptoms, and
determining, from said evaluation, an indication of the patient belonging to a pre-ictal or inter-ictal state, said indication being obtained by implementation of a model trained according to the training method disclosed above, and,
if the patient is determined as belonging to a pre-ictal state, generating an alert signal.

In embodiments, the memory further stores the trained model and the step of determining an indication of the patient belonging to a pre-ictal or inter-ictal state comprises implementation by the computing device of the trained model on the patient's evaluation.

In embodiments, the computing device is further configured for receiving an input of the patient regarding occurrence or not of a seizure in a predefined period of time following an evaluation, and for updating the training of the model according to the patient's data.

In embodiments, the computing devices comprises a first device configured to communicate with a remote server storing the trained model, wherein the first device is configured for receiving said evaluation from the patient, and transmitting said evaluation to the remote server, and said remote server is configured for implementing the trained model on said evaluation in order to obtain said indication of the patient belonging to a pre-ictal or inter-ictal state.

The claimed method allows training a prediction model which is configured to determine whether a patient, who provides a self-evaluation on the intensity of a plurality of prodromal symptoms, belongs to an inter-ictal state or a pre-ictal state, where a pre-ictal state is a state preceding the occurrence of a seizure in a determined period of time following the evaluation, for instance 12 or 24 h.

The method, based on a Support Vector Machine classifier, provides increased prediction performances, which can be further enhanced by selecting a subset of the most relevant prodromal symptoms. Those most relevant symptoms can differ from one person to another, and hence the selection of the most relevant symptoms can be performed on a person-by-person basis.

In some embodiments, the evaluation by a patient of its prodromal symptoms can be performed regularly, for instance daily, on a computing device which can obtain a seizure prediction based on this evaluation. The same device may be used to take advantage from the evaluation data regularly provided by that patient, in order to update the training of the prediction model and to improve its performance regarding that patient. The computing device may be a personal device such as a mobile phone or a digital tablet.

### BRIEF DESCRIPTION OF THE DRAWINGS

Other features and advantages of the invention will be apparent from the following detailed description given by way of non-limiting example, with reference to the accompanying drawings, in which:

FIG. 1 represents a training device according to an embodiment of the invention,

FIG. 2a represents a method for training a prediction model,

FIG. 2b represents a method for implementing the prediction model,

FIG. 3a represents an embodiment of a computing device collecting evaluation data from a patient, and implementing the prediction model,

FIG. 3b represents another embodiment of a computing device collecting evaluation data from a patient and communicating with a remote server for implementation of the prediction model.

### DETAILED DESCRIPTION OF AT LEAST ONE EMBODIMENT

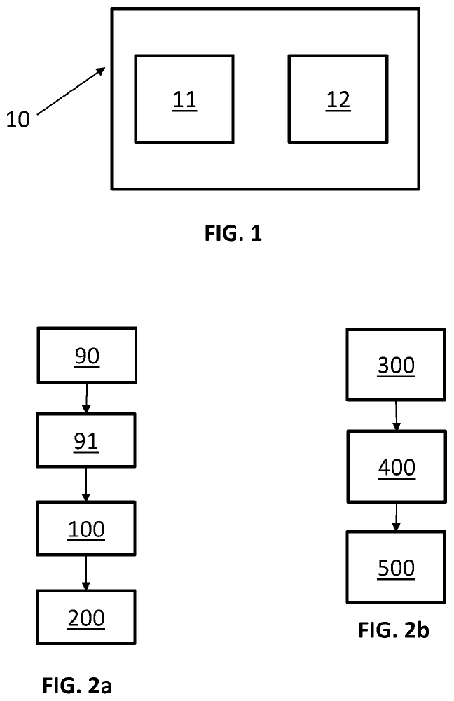

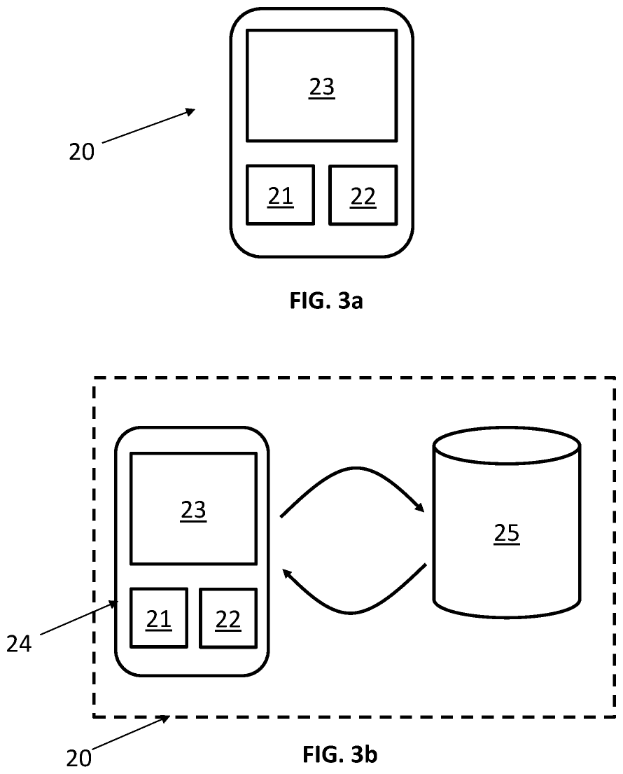

With reference to FIG. 1, it is shown a training device 10 configured for implementing the training of a prediction model for predicting occurrence of an epileptic seizure. The training device 10 comprises a computer 11, for instance a processor or microprocessor, including for instance a Computer Processing Unit CPU or a Graphical Processing Unit GPU. The training device further includes a memory 12, storing code instructions executed by the computer 11 for implementing the training method, and also storing a training dataset. The memory also stores the parameters of the prediction model and their updates during training of the model.

The training dataset comprises data inputs obtained from a plurality of epileptic patients, where each data input corresponds to the evaluation, from a patient, of the intensity of each of a pre-defined set of prodromal symptoms. The pre-defined set of prodromal symptoms is thus the same for all patients and all evaluations of a given patient.

With reference to FIG. 2a, the training method may include a preliminary step 90 of acquiring such training dataset. This acquisition may be performed by receiving evaluation from patients, on a regular basis, for instance every day or twice a day, about the intensity of each prodromal symptom of the pre-defined set. For instance, each patient may be interrogated by a person, or may fill out an auto-questionnaire, which can be presented in a paper or digital interface.

In embodiments, the intensity of each prodromal symptom is evaluated on a scale of between two and ten points, and preferably between three and ten points. This enables an individual to express in a more detailed manner the intensity of a prodromal symptom rather than merely expressing presence or absence of this symptom. In an embodiment, each symptom is evaluated by a patient on a four-point scale, in particular a four-point Likert scale, in which the four points express progressive intensity of each symptom. An intensity level may for instance be selected among the following propositions: “Not at all”, “a little bit”, “quite so” and “very much so”.

According to this example, the evaluation by a patient may for instance be converted during a conversion step 91 into a numerical vector where each symptom is represented by one number of the set {0, 1, 2 3} according to the level selected by the patient. For example an evaluation corresponding to Table 2 may be converted into the following vector: [1 3 2]

TABLE 2

| | | | |
|---|---|---|---|
| Not | | | Very |
| at all | A little bit | Quite so | much so |

| | | | |
|---|---|---|---|
| Headache | X | | |
| Nausea | | | X |
| Blurred vision | | X | |

The pre-defined set of prodromal symptoms preferably comprises a plurality of items, for instance at least 24 different symptoms. The set of prodromal symptoms may include symptoms selected among the following groups:
Cognitive symptoms,
Sensory symptoms,
Emotional symptoms,
Physical symptoms.

Cognitive prodromal symptoms may include one, several or all of the following symptoms:
Troubles in concentrating,
Troubles in understanding,
Troubles in talking,
Troubles in reading,
Troubles in writing.

Sensory symptoms can include one, several or all of the following symptoms:
Blurred vision,
Light sensitivity,
Noise sensitivity,
Hearing Impairment,
Tinnitus

Emotional symptoms may include one, several or all of the following symptoms:
Bad mood,
Irritability,

Physical symptoms may include one, several or all of the following symptoms:
Clumsiness
Tremor,
Urge to urinate,
Spinning head,
Nausea,
Headache,
Thirst,
Hunger,
Funny feeling,
Fatigue.

In embodiments, the pre-defined set of prodromal symptoms may include at least one, or at least two symptoms from each group. In embodiments, the pre-defined set of prodromal symptoms includes all the symptoms recited above of each group. This set may also include an additional prodromal symptom corresponding to: i) the self-evaluation by the patient about her/his likelihood for an epileptic seizure occurrence during the day; ii) their anxiety level; iii) the last night's sleep quality and/or iv) the presence/absence of premenstrual syndromes.

In addition, each data input of the training dataset is associated to an indication of said patient belonging to a pre-ictal state or inter-ictal state at the time of the evaluation, wherein:
pre-ictal state is defined as a state preceding occurrence of at least one epileptic seizure during a determined period of time T following the evaluation, and
inter-ictal state is defined as a state preceding no occurrence of epileptic seizure during the period of time T following the evaluation.

In embodiments, each patient is monitored during the determined period of time T following the evaluation to detect occurrence or not of a seizure. If this monitoring is performed in a clinical environment, it may be performed together with continuous EEG or video-EEG monitoring during the period of time following the evaluation. Detection of an epileptic seizure through EEG or video-EEG is well known in the field of epilepsy. If, during this period of time T, no seizure occurs, it is considered that the patient was in an inter-ictal state at the time of evaluation, whereas if at least one seizure occurs, it is considered that the patient belonged to a pre-ictal state.

Alternatively, the indication of the patient belonging to a pre-ictal state or inter-ictal state at the time of the evaluation can be provided retroactively by the patient. The patient can indeed indicate occurrence of a crisis and the date and time of the crisis.

By establishing a correlation between the date and time of the crisis and the time of acquisition of the evaluation by the patients about the prodromal symptoms, the state in which the patient was at the time of the evaluation can be determined.

In embodiments, the fixed period of time T for detecting occurrence of a seizure is comprised between 5 and 36 hours. This fixed period of time may for instance be equal to either 6 hours, 12 hours or 24 hours.

The prediction model is a non-linear binary classification model, for instance a Support-Vector Machine (SVM) model with a non-linear kernel, adapted to project the input data into a higher dimensional space where classification of data between the inter-ictal state and pre-ictal state can be performed. SVM models can be chosen because of their robustness for modelling complex data without any prior assumption under the underlying distribution. Moreover, SVM models do not require huge amounts of training data. In an embodiment, the classification model is a SVM model with a Gaussian kernel.

The classification model is configured to receive as input a data input of a patient (in the form of a vector), which is the evaluation by the patient of the intensity of each of the predefined set of prodromal symptoms, and to output an indication of the patient belonging either to a pre-ictal or inter-ictal state. The definition of pre-ictal or inter-ictal state is here the same as the definition given for the indication associated to a data input of the patient. In particular, the prediction that the classification model is trained to perform is applicable to the same period of time of observation following the evaluation than the period of time T observed in the training dataset.

In addition, where the classification model is a SVM model, it may be configured to also output the distance between an input data (once transformed using the selected kernel) and the hyperplane separating the input data between the two classes formed by the inter-ictal state and the pre-ictal state. This enables determining a confidence index based on said distance upon later implementation of the prediction model.

The training method implemented by the training device 10 thus comprises a supervised training 100 of the prediction model on the training data set, said supervised training comprising a training phase on a part of the training data set, for instance between 50 and 80% thereof, and a testing phase using a k-fold cross-validation procedure on the remaining data (50 to 20%).

In embodiments, the supervised training 100 may be followed by a step 200 of selecting, among the predefined set of prodromal symptoms, a subset of symptoms that improves the model performances. This step may be implemented by evaluating the performance of the prediction model with the full training set 100, removing one symptom, re-training the model on the basis of the new set (without the removed symptom), and revaluating the performances of the prediction model without this symptom. The symptom which removal most increases performance of the classification is removed, and the same may be implemented until no increase in performance of the model is noticed. The performance of the prediction model may be measured by estimating at least one classical performance attribute such as:
AUC (Area Under the Curve) value of the receiver operating characteristic (ROC), ranging from 0.5 (random classification) to 1 (perfect classification),
Sensitivity (proportion of correctly predicted true positives),
Specificity (proportion of correctly predicted true negatives),
Accuracy (proportion of true results, either positive or negatives). As it may be biased towards the majority class, we can consider the precision (the fraction of relevant instances among the detected instances)

In embodiments, step 200 may also be implemented with data obtained from a single patient, in order to maximize the performances of the model for said patient. This allows taking into account the fact that some prodromal symptoms may yield more reliable and accurate prediction of the occurrence of a seizure for some patients, while others symptoms may provide better prediction performances with other patients.

Once the prediction model has been trained, it may be implemented by a computing device 20, which may be distinct from the training device, on newly acquired data. This computing unit comprises a processing unit 21, for instance a processor, a CPU, a GPU, etc. and a memory 22 storing code instructions for execution by the processing unit.

Prediction of occurrence of a seizure may then be implemented by:
Receiving 300 an evaluation, by a patient, of the intensity of each prodromal symptom among the set of symptoms that has been eventually selected at the end of the training of the prediction model. This set may correspond to the pre-defined set of symptoms, but may also correspond to only a subset of symptoms if there has been a step 200 of selection of the most reliable symptoms.
Determining 400, according to the received evaluation, an indication whether the patient is in a pre-ictal state, meaning that occurrence of a seizure is predicted, or in an inter-ictal state, whereby said indication is obtained by implementing the trained prediction model.

As regards the step 300 of receiving an evaluation, this step may be implemented by obtaining data corresponding to said evaluation at the processing unit, by loading from a memory, downloading from a network, etc.

In other embodiments, the computing device may also comprise a Human-Machine Interface 23, allowing the patient to directly fill in his evaluation. The Human-Machine Interface can for instance be a display screen showing the questionnaire to be filled in. The display can further be tactile, or the Human-Machine Interface can also include a keyboard or any other interface allowing the patient to provides its evaluation.

In the case where the patient is determined as being in a pre-ictal state, an alert signal can be emitted during a step 500. In embodiments, this signal may be visual a message, picture, symbol, color, or any other visual warning displayed by the Human-Machine Interface 23, inviting the patient to avoid activities which may be dangerous upon occurrence of a seizure, such as driving. The signal may alternatively or in complement include an audible signal, such as an audio message, signal, or tone that is prompted by the Human-Machine Interface 23. In other embodiments, the signal may be sent to the patient's doctor (neurologist), for instance in the form of a warning message sent for instance by email, SMS, or through a notification prompted by an application.

In embodiments, if the classifier is a SVM model configured for outputting the distance between the separation hyperplane and the received input data, the prediction may also comprises evaluating a confidence index associated with the prediction. This confidence index may be qualitative, for instance selected among values such as “low confidence, average confidence, high confidence”. Said confidence index may be displayed or sent along with the alert signal.

The steps of receiving an evaluation and implementing the prediction model may be performed regularly, with a time interval between two evaluations corresponding to the fixed period of time for which the model has been trained. For instance, if the inter-ictal and pre-ictal states are defined for a period of time of 24 hours following an evaluation, the steps of receiving an evaluation and implementing the model may be performed every 24 hours.

Accordingly, the method is particularly convenient for the patients since the implementation of the prediction model does not require an EEG recording or any other recording of brain activity, as the classification performed by the prediction model is established based on input data comprising only evaluation of the set of prodromal symptoms, exclusive of EEG recording.

In an embodiment schematically represented in FIG. 2a, the computing device 20 may be a personal terminal of the patient, such as a mobile phone (e.g. smartphone), personal computer or digital tablet, on which a dedicated software application is downloading for displaying the questionnaire, and receiving the inputs from the patient. The memory 22 of the personal device 20 may store the trained prediction model. In that case, the personal device 20 can itself implement the trained model and predict the state of the patient.

Furthermore, the personal device 20 may also be used to enhance the training of the model in order to adapt it to each patient's specificities. To do so, the computing device may be further configured to receive indication from a patient regarding occurrence or not of a seizure during the determined period of time following the last evaluation.

For instance, if the prediction model has been trained to predict occurrence of a seizure in a period of time of 24 hours following an evaluation, the computing device may be configured to interrogate the patient every day at the same time, to receive simultaneously:
its evaluation regarding the intensity of the prodromal symptoms, and
whether or not the patient underwent a seizure in the last 24 hours.

Accurate and regular data relative to the patient can thus be acquired and the computing device may then update the training of the prediction model according to said data.

According to another embodiment shown in FIG. 2b, the computing device 20 includes a first device 24, e.g. a personal device of the patient, such as a mobile phone, personal computer or digital tablet, used to acquire the evaluation data, said first device being configured to communicate with a remote server 25 having its own processor and memory (not shown) storing the trained prediction model. According to this configuration, the server 25 may be able to communicate with each one of a plurality of devices personal to respective patients, and to acquire evaluation data from those devices.

According to this embodiment, the first device 24 may receive the evaluation regarding the intensity of the prodromal symptoms, and transfer said evaluation to the remote server 25, which in turns implements the trained prediction model to obtain the classification of the patient as being in a pre-ictal or inter-ictal state. This indication may then be transferred back to the first device 23, or it may be transferred to a doctor in charge of the patient, in particular if a pre-ictal state is predicted.

If the first device(s) 24 also transfers data regarding actual absence or occurrence of seizures to the remote server 25, the latter may also perform regular updates of the training of the prediction model.

### Example

A study was conducted taking into account, as the pre-defined set of prodromal symptoms, the 22 symptoms recited above in addition to the self-prediction of seizure. Each item was scored using a four-point Likert scale from “not at all” to “very much so”.

Anxiety level was also evaluated using the validated Sate-Trait Anxiety Inventory (STAI) form Y-1, which includes 20 questions with a four-point Likert scale and scores range from 20 (low) to 80 (high anxiety). These scores where then converted using the same four-point scale as other prodromal symptoms: 0 [20-35], 1 [36-50], 2 [51-65], and 3 [66-80].

A set of twenty-four patients underwent a continuous video-EEG monitoring, and where asked to fill a questionnaire every morning. The mean age of the patients was 35 years (min 22, max 54), and 13 patients were women. The mean duration of video-EEG monitoring was 10.3 days (min 2 days, max 21 days). Patients had mainly temporal focal epilepsy (N=14, 58.3%) and a mean number of seizures of 3.8 during the study.

Every daily questionnaire was then classified into either a pre-ictal group (58 cases) or inter-ictal group (190 cases).

Classification was performed using a SVM classifier with Gaussian kernel. The training of the model included a training phase over 70% of the training dataset and a validation phase using a cross-validation used with 10000 folds.

In order to prevent a biased model related to class-imbalance data (more cases in inter-ictal group than pre-ictal group), or overoptimistic predictions, Synthetic Minority Oversampling Technique (SMOTE) was performed in order to oversample points of the minority class, within each fold of cross-validation.

The SVM classifier was first evaluated with the whole set of prodromal symptoms, then less relevant symptoms were removed one by one by a pruning procedure based on AUC values after removal of each symptom. Accordingly, 11 symptoms were removed, after what further removal induced decrease in classification performance.

For comparison purposes, the results achieved with the SVM classifier were compared with results obtained from a Fisher's classifier (linear classification model).

A linear prediction model based on the Fisher's classifier yielded poor results using a combination of the 24 symptoms (AUC=0.55; C.I. [0.40-0.69]) and no improvements were obtained by the feature removal procedure (AUC=0.59; C.I. [0.44-0.73]).

On the other hand, the SVM-based predictions using the combination of all the 24 symptoms provided a much better prediction performance (AUC=0.72, C.I. [0.61-0.81]), which was slightly improved by selecting the most relevant symptoms over the whole population of patients (AUC=0.80, C.I. [0.69-0.88]).

1. A method for training a model for predicting occurrence of an epileptic seizure, the method being implemented by a training device comprising a computer and a memory storing a training dataset,
wherein the training dataset comprises data inputs obtained from a plurality of epileptic patients, each data input comprising an evaluation, by a patient, of the intensity of each of a predefined set of prodromal symptoms, each data input being further associated to an indication of said patient belonging to a pre-ictal or inter-ictal state at the time of the evaluation,
wherein a pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation,
and the method comprises performing supervised training, over the training dataset, of a nonlinear binary classification model configured to receive as input the evaluation, by a patient, of the intensity of each prodromal symptom among the predefined set of prodromal symptoms, and to output a classification of said patient belonging either to a pre-ictal or inter-ictal state.

2. The method according to claim 1, wherein the intensity of each prodromal symptom is evaluated on a four-points Likert scale.

3. The method according claim 1, wherein the fixed period of time following the evaluation is between 6 h and 24 h.

4. The method according to claim 1, wherein the prediction model is a SVM classifier with a Gaussian kernel.

5. The method according to claim 1, further comprising a step of selecting, among the predefined set of prodromal symptoms, a subset of prodromal symptoms maximizing the performances of the model over the data inputs relative to a specific patient.

6. The method according to claim 4, wherein the training of the SVM classifier comprises determining a hyperplane separating input data into two classes corresponding respectively to a pre-ictal state and an inter-ictal state, and the SVM classifier is further configured to output an indication of a distance between each piece of input data and said hyperplane.

7. A training device comprising a computer and a memory storing a training dataset,
wherein the training dataset comprises data inputs obtained from a plurality of epileptic patients, each data input comprising an evaluation, by a patient, of the intensity of each of a predefined set of prodromal symptoms, and an indication of said patient belonging to a pre-ictal or inter-ictal state at the time of the evaluation,
wherein a pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation,
wherein the training device is configured for implementing the method according to claim 1.

8. A computer-implemented nonlinear binary classification model, configured to receive as input an evaluation by a patient of the intensity of each one of a set of prodromal symptoms, and to output an indication of the patient belonging to a pre-ictal state or inter-ictal state, wherein the pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation, wherein the computer-implemented nonlinear binary classification model has been trained by implementing the method according to claim 1.

9. A computer-implemented method for predicting occurrence of an epileptic seizure, comprising:
receiving an evaluation by a patient of the intensity of each one of a set of prodromal symptoms,
implementing a model according to claim 8 on said evaluation, to output an indication of the patient belonging to a pre-ictal or inter-ictal state, wherein a pre-ictal state is defined as a state preceding occurrence of at least one epileptic seizure during a fixed period of time following the evaluation, and inter-ictal state is defined as a state preceding no occurrence of epileptic seizure during the fixed period of time following the evaluation.

10. The computer-implemented method for predicting occurrence of an epileptic seizure according to claim 9, wherein the model is a SVM classifier with a Gaussian kernel, the model has been trained according to a training comprising determining a hyperplane separating input data into two classes corresponding respectively to a pre-ictal state and an inter-ictal state, and the SVM classifier is further configured to output an indication of a distance between each piece of input data and said hyperplane, and the method further comprises computing a confidence index associated to the classification of the patient as belonging to the pre-ictal or inter-ictal state, based on the distance between a vector representative of the patient's evaluation and the hyperplane determined during training of the model, and outputting said confidence index.

11. A non-transitory computer-readable storage medium having stored thereon code instructions for implementing the method according to claim 1, when it is implemented by a computer.

12. A computing device comprising a processing unit, a memory storing code instructions executed by the processing unit, and a Human-Machine Interface, wherein the computing device is configured for:
receiving, from a patient, an evaluation of the intensity of each of a plurality of prodromal symptoms, and
determining, from said evaluation, an indication of the patient belonging to a pre-ictal or inter-ictal state, said indication being obtained by implementation of a model according to claim 8, and,
if the patient is determined as belonging to a pre-ictal state, generating an alert signal.

13. The computing device according to claim 12, wherein the memory further stores the trained model and the step of determining an indication of the patient belonging to a pre-ictal or inter-ictal state comprises implementation by the computing device of the trained model on the patient's evaluation.

14. The computing device according to claim 13, wherein the computing device is further configured for receiving an input of the patient regarding occurrence or not of a seizure in a predefined period of time following an evaluation, and for updating the training of the model according to the patient's data.

15. The computing device according to claim 12, comprising a first device configured to communicate with a remote server storing the trained model, wherein the first device is configured for receiving said evaluation from the patient, and transmitting said evaluation to the remote server, and said remote server is configured for implementing the trained model on said evaluation in order to obtain said indication of the patient belonging to a pre-ictal or inter-ictal state.

16. A non-transitory computer-readable storage medium having stored thereon code instructions for implementing the method according to claim 9, when it is implemented by a computer.
